(19) United States
(12) Patent Application Publication
(10) Pub. No.: US 2014/0134122 A1
(43) Pub. Date: May 15, 2014

(54) SKIN CARE COMPOSITIONS INCLUDING SORBED COMPOUNDS AND METHODS FOR MAKING SAME

(71) Applicant: THE DIAL CORPORATION, Scottsdale, AZ (US)

(72) Inventors: Danielle Lipski, Phoenix, AZ (US); Travis T. Yarlagadda, Phoenix, AZ (US)

(73) Assignee: The Dial Corporation, Scottsdale, AZ (US)

(21) Appl. No.: 13/677,426

(22) Filed: Nov. 15, 2012

(51) Int. Cl. A61K 8/46; A61K 8/02; A61Q 15/00; A61K 8/25

(52) U.S. Cl. CPC A61K 8/46; A61K 8/25; A61K 8/0279; A61Q 15/00
USPC 424/65

(57) ABSTRACT

Skin care compositions, antiperspirant compositions and methods for making such compositions are provided herein. In one embodiment, an antiperspirant composition includes an active antiperspirant compound. A sorbent is dispersed throughout the composition. The composition further includes a hair growth inhibiting agent sorbed by the sorbent.

### TECHNICAL FIELD

The technical field relates generally to skin care compositions and methods of making antiperspirant compositions, and more particularly relates to compositions and methods for making compositions that include sorbed hydrated compounds in an anhydrous base.

### BACKGROUND

Antiperspirant and deodorant compositions are well known skin care products used to prevent or eliminate perspiration and body odor caused by perspiration. The compositions come in a variety of forms and may be formulated, for example, into aerosols, pumps, sprays, liquids, roll-ons, lotions, creams, sticks, and soft solids, etc.

Various types of stick antiperspirant compositions are desired by a large majority of the population because of their ease of application and the presence of active antiperspirant compounds, e.g. antiperspirant salts, that prevent the secretion of perspiration and its accompanying odors. In one type, an antiperspirant salt is suspended in an anhydrous vehicle often including a solid water-insoluble wax.

In addition to inhibiting perspiration and body odor at the underarm, a portion of the population actively removes hair from the underarm. While mammalia hair provides environmental protection, that function has largely been lost in humans, in whom hair is kept or removed from various parts of the body essentially for cosmetic reasons.

Various procedures have been employed to remove unwanted hair, including shaving, electrolysis, depilatory creams or lotions, waxing, and plucking. These conventional procedures generally have drawbacks associated with them. Shaving, for instance, can cause nicks and cuts, and can leave a perception of an increase in the rate of hair regrowth. Shaving also can leave undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair.

Accordingly, it is desirable to provide skin care compositions that contain sorbed agents to prevent unwanted interaction between the sorbed agents and the composition base. Further, it is desirable to provide antiperspirant compositions that exhibit strong antiperspirant efficacy and inhibit hair growth. Further, it is desirable to provide antiperspirant compositions that release hair growth inhibiting agents over time through contact with perspiration. In addition, it is desirable to provide methods for making such antiperspirant compositions. Furthermore, other desirable features and characteristics will become apparent from the subsequent detailed description and the appended claims, taken in conjunction with the accompanying drawings and the foregoing technical field and background.

### BRIEF SUMMARY

Skin care compositions, antiperspirant compositions and methods for making such compositions are provided herein. In an exemplary embodiment, an antiperspirant composition includes an active antiperspirant compound. A sorbent is dispersed throughout the composition. The composition further includes a hair growth inhibiting agent sorbed by the sorbent.

In accordance with another exemplary embodiment, a skin care composition is provided. The skin care composition includes an anhydrous base. Further, the skin care composition includes a sorbent dispersed throughout the anhydrous base. Also, a hydrated compound is sorbed by the sorbent. Interaction between the anhydrous base and the hydrated compound is prevented by sorbing the hydrated compound into or onto the sorbent.

In accordance with another exemplary embodiment, a method for making an antiperspirant composition is provided. The method includes sorbing a hair growth inhibiting agent with a sorbent to form a sorbed hair growth inhibiting agent. The method forms an antiperspirant mixture with an active antiperspirant compound. Further, the method includes dispersing the sorbed hair growth inhibiting agent into the antiperspirant mixture.

### BRIEF DESCRIPTION OF THE DRAWING

Embodiments of skin care compositions and methods for making skin care compositions that include sorbed hydrated compounds will hereinafter be described in conjunction with the following drawing figure wherein:

FIG. 1 is a perspective view of a skin care composition in product form in accordance with an exemplary embodiment.

### DETAILED DESCRIPTION

The following detailed description is merely exemplary in nature and is not intended to limit the skin care compositions and methods for making skin care compositions as claimed herein. Furthermore, there is no intention to be bound by any expressed or implied theory presented in the preceding technical field, background or brief summary, or in the following detailed description.

The various embodiments contemplated herein relate to skin care compositions that include hydrated compounds sorbed onto or into a sorbent. As a result, negative interactions between hydrated compounds and anhydrous portions of the skin care composition are avoided. A particular embodiment provides antiperspirant compositions that exhibit strong antiperspirant efficacy and inhibit the growth of hair, and methods for making such antiperspirant compositions. Unlike the prior art, the exemplary embodiments herein include an antiperspirant composition with an active antiperspirant compound and with a hair growth inhibiting agent that is sorbed by a sorbent such as silica particles. As used herein, “sorbed” means held, as by absorption into or adsorption onto, by another substance. In other words, the hair growth inhibiting agent may be absorbed into and/or adsorbed onto the sorbent.

The active antiperspirant compound is effective to prevent the secretion of perspiration and/or accompanying odors. The hair growth inhibiting agent is effective to inhibit the growth of hair at the site on the user where it is applied, such as on the underarm. The hair growth inhibiting agent is sorbed onto silica particles to delay its release until the silica releases the inhibiting agent, such as when contacted by perspiration after application by the user. Further, by sorbing the hair growth inhibiting agent onto or into the sorbent, interaction between the hair growth inhibiting agent and other components in the composition are prevented. For example, the active antiperspirant compound does not react with the hair growth inhibiting agent and instead retains its activity. Also, by preventing interaction with the hair growth inhibiting agent, the composition retains its desired properties for application, and the composition retains the desired feel and efficacy for delivery to the user. Upon contact with perspiration after application by the user, the silica will release the hair growth inhibiting agent, and the hair growth inhibiting agent contacts the hair and follicle to inhibit hair growth.

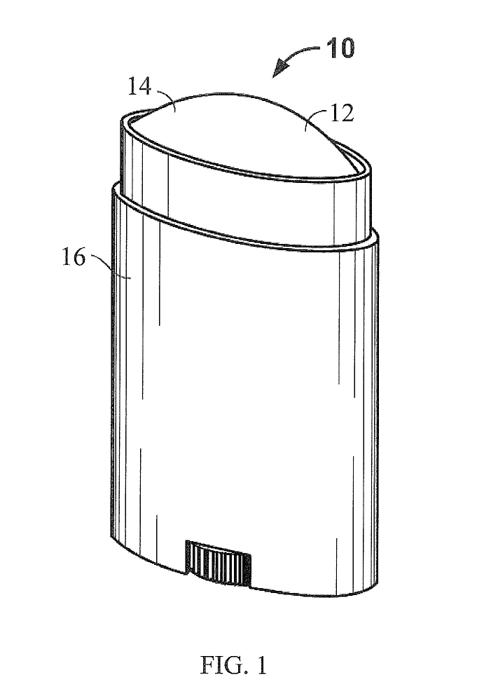

Referring to FIG. 1, skin care product 10, such as an antiperspirant product, is provided in accordance with an exemplary embodiment. The antiperspirant product 10 comprises an antiperspirant composition 12, such as a solid wax antiperspirant formulation. As illustrated, the antiperspirant composition 12 has an application surface 14 that is substantially dome-shaped and that is configured to be applied to skin, such as, for example, an underarm. The antiperspirant product 10 may also comprise a container or dispenser 16 for dispensing the antiperspirant composition 12 to the skin. While illustrated as a solid wax formation, the antiperspirant composition can be in any number of anhydrous antiperspirants or antiperspirants that have a distinct anhydrous phase such as an emulsion, with forms including solid stick, aerosol, roll on, gel, etc.

The antiperspirant composition contains at least one active ingredient or active antiperspirant compound, which typically includes metal salts. The metal salts are believed to reduce perspiration by diffusing through the sweat ducts of eccrine glands and apocrine glands and hydrolyzing in the sweat ducts. They combine with proteins to form an amorphous metal hydroxide agglomerate that plugs the sweat ducts so that perspiration cannot diffuse to the skin surface. Some active antiperspirant compounds that may be used include astringent metallic salts, such as inorganic and organic salts of aluminum, zirconium, and zinc, as well as mixtures thereof. Some examples are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} masking layer (H₂O), wherein a is from 2 to about 5; the sum of a and b is about 6; masking layer is from about 1 to about 6; and wherein a, b, and masking layer may have non-integer values. Exemplary zirconium salts include those having the general formula ZrO(OH)₂₋ₐClₐ masking layer (H₂O), wherein a is from about 1.5 to about 1.87, masking layer is from about 1 to about 7, and wherein a and masking layer may both have non-integer values. Some zirconium salt examples are those complexes that additionally contain aluminum and glycine, commonly known as ZAG complexes. These ZAG complexes contain aluminum chlorohydroxide and zironyl hydroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof.

The active antiperspirant compound is preferably in a perspiration-reducing effective amount. In one embodiment, the antiperspirant composition 12 comprises an active antiperspirant compound present in the amount of from about 5 to about 30 weight percent. As used herein, weight percent or wt. % of an antiperspirant salt is calculated as anhydrous weight percent in accordance with the U.S.P. method, as is known in the art. This calculation excludes any bound water and glycine.

The antiperspirant composition 12 contains a hair growth inhibiting agent that is effective to inhibit the growth of hair. The hair growth inhibiting agent is sorbed by a sorbent, such as silica particles. In an exemplary embodiment, the silica particles sorbed with the hair growth inhibiting agent are present in an amount of from about 0.0 to about 6.0 wt. % of the antiperspirant composition 12. In an exemplary embodiment, the hair growth inhibiting agent is present in an amount of from about 0.0 to about 3.0 wt. % of the antiperspirant composition 12.

In an exemplary embodiment, the silica particles are hydrophilic. “Hydrophilic” silica particles, as the term is used herein, encompasses silica particles having varying levels or degrees of hydrophilicity. The silica particles may have a BET surface area (ASTM D6556-07) of about 2 m²/g to about 400 m²/g, for example, from about 2 m²/g to about 5 m²/g, from about 5 m²/g to about 10 m²/g, from about 10 m²/g to about 25 m²/g, from about 25 m²/g to about 50 m²/g, from about 50 m²/g to about 100 m²/g, from about 100 m²/g to about 150 m²/g, from about 150 m²/g to about 200 m²/g, from about 200 m²/g to about 250 m²/g, from about 250 m²/g to about 300 m²/g, from about 300 m²/g to about 350 m²/g, or from 350 m²/g to about 400 m²/g. In an exemplary embodiment of the invention the silica is hydrophilic forms of precipitated silica having a BET surface area from 50 to 1000 m²/g, preferably from 100 to 700 m²/g, more preferably 150 to 600 m²/g, a particle size d₅₀, determined by laser diffraction, from 2 to 130 μm, preferably 3 to 20 μm

Preferably, the silica particles are porous and hydrophilic such that when the user perspires, the perspiration is readily absorbed by the silica particles, displacing or driving out the cooling sensation agent and the solubilizer from the silica particles.

In an exemplary embodiment, the sorbent includes porous particles and the hair growth inhibiting agent is absorbed into pores in the particles and/or onto the surface of the particles. For example, the sorbent may be porous hydrophilic silica particles.

The hair growth inhibiting agents may include thioglycolate salts or thioglycolic acids such as sodium, potassium, magnesium, calcium, beryllium, strontium, zinc, monoethanolamine, ammonium, tetralkylammonium, imidazolium, pyridinium, phosphonium or glyceryl thioglycolate salts, or mixtures thereof, which may include dianion forms of thioglycolate. More preferably, the hair growth inhibiting agent comprises at least one of sodium, potassium, magnesium or calcium thioglycolate, or mixtures thereof. In an exemplary embodiment, the hair growth inhibiting agent comprises potassium thioglycolate, calcium thioglycolate, or mixtures thereof. In a preferred embodiment, the concentration of the thioglycolate salt (which may include all species in the deprotonation equilibrium system) is from 0.0% to 6.0%, more preferably from 0.1% to 3.0% of the antiperspirant composition.

In some embodiments, the antiperspirant composition may further comprise a monovalent cation, preferably a monovalent metal cation, which may increase the dissociation of thioglycolate salts. The monovalent cations such as those derived from monovalent cation-containing salts are able to displace the cation of the thioglycolate salt and further enhance dissociation of the thioglycolate salt. This increases the amount of deprotonated thioglycolate formed from the thioglycolate salt and therefore increases the effectiveness of the hair growth inhibiting agent. Sources of monovalent cations include potassium, sodium, lithium, ammonium, tetraalkyl ammonium and imidazolium salts, which may be a component of another ingredient, for example a thickening system or skin care active. Preferred sources of monovalent cations include potassium and sodium salts.

In order to further enhance the safety of the resulting product, it may be advantageous to limit the amount of monovalent cations, such as monovalent metal cations, to which the skin is exposed when the antiperspirant composition is used, although a small quantity may improve the efficacy of the hair growth inhibiting agent. Advantageously, the quantity of monovalent cations (or monovalent metal cations in the preferred embodiment above) per unit area of the aforementioned coated region is less than about 5.10×10⁻⁴ mol/cm², for example, less than about 3×10 mol/cm⁻², such as from about 1×10⁻⁹ mol/cm² to about 1.5×10⁻⁴ mol/cm², for example from about 2.50×10⁻⁸ mol/cm² to about 6.65×10⁻⁵ mol/cm², such as from about 6×10⁻⁷ mol/cm² to about 4.5×10⁻⁵ mol/cm². The selection of hair growth reducing agent and other ingredients may be based on the quantity of monovalent cations or monovalent metal cations achieved.

Limiting the quantity of monovalent ion present in the hair growth inhibiting agent may prevent skin irritation but also limits the quantity of thioglycolate salt that may be present in a formula if monovalent ion-containing thioglycolate salts or bases are used. Accordingly, in an advantageous embodiment, the hair growth inhibiting agent comprises a divalent cation, such as a divalent metal cation, and wherein the thioglycolate salt, a buffering base (if present) or both comprises a divalent cation, or a divalent metal cation in order to enable the inclusion of additional depilatory active. In another exemplary embodiment, the thioglycolate salt comprises a divalent metal cation. Applicants have established that thioglycolate salts comprising monovalent metal cations, such as potassium thioglycolate, are effective at removing hair from the skin, even at low doses, but may expose the skin tissue to harsh chemical conditions, resulting in irritation. On the other hand, thioglycolate salts comprising divalent metal cations, such as calcium thioglycolate, are relatively non-irritating to the skin.

In a hair growth inhibiting agent comprising a mixture of monovalent and divalent ions, controlling the ratio of divalent ions to monovalent ions may also improve the safety characteristics of the antiperspirant composition. Increasing the concentration of divalent ions relative to the concentration of monovalent ions increases the likelihood that any particular hair growth reducing active species is associated with a divalent ion, rather than the more irritating monovalent ions. On the other hand, increasing the concentration of monovalent ions increases the effectiveness of the hair growth inhibiting agent. Accordingly, in an alternative embodiment the ratio of the concentration of divalent ions to the concentration of monovalent ions present in the hair growth inhibiting agent is advantageously in the range of from about 400:1 to about 0.02:1, for example, from about 200:1 to about 0.1:1, for example about 60:1 to about 0.3:1, for example from about 20:1 to about 0.5:1, such as from about 15:1 to about 1:1.

It is herein disclosed that sorbing the hair growth inhibiting agent with the silica particles allows the hair growth inhibiting agent to be held for release from the silica particles upon introduction to a selected environment, such as a user's underarm. For example, the silica particles are configured to release upon contact with perspiration. Therefore, the hair growth inhibiting agent may be held by each sorbent particle until it is at least partially dissolved by the perspiration. Upon partial dissolution of each sorbent particle, the hair growth inhibiting agent is released from the dissolved particle, and may interact with a hair follicle it contacts to inhibit growth of hair therefrom.

The antiperspirant composition may further comprise an anhydrous, hydrophobic vehicle, which includes a volatile silicone and/or a high melting component. In an exemplary embodiment, the active antiperspirant compound is suspended in the anhydrous, hydrophobic vehicle.

For use as an antiperspirant stick, the high melting components may include any suitable material that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, such as from about 2 to about 15 wt. %, of the antiperspirant composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The antiperspirant composition contemplated herein also may comprise additives, such as those used in conventional antiperspirants. For example, in addition to antiperspirant efficacy, the antiperspirant composition 12 may comprise additives that cause the antiperspirant composition 12 to exhibit long-lasting fragrance, odor protection, bacteria control, and/or another desired purpose and/or function. These additives include, but are not limited to, fragrances, including encapsulated fragrances, dyes, pigments, preservatives, antioxidants, moisturizers, and the like. These optional ingredients can be included in an amount of from about 0 to about 20 wt. % of the antiperspirant composition 12.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as particulate polyolefins, talcum materials, colorants and preservatives may also be included as desired. For example, the antiperspirant composition 12 may include up to about 5% fragrance or about 2% colorant by weight.

As noted above, in addition to an active antiperspirant compound, the antiperspirant composition 12 includes a component or components that cause it to exhibit or impart a desired function or purpose in addition to antiperspirant efficacy. For example, the antiperspirant composition 12 includes hair growth inhibiting agents, such as potassium or calcium thioglycolate.

In an exemplary embodiment, the hair growth inhibiting agent is hydrated or in aqueous form and the antiperspirant composition is anhydrous or in non-aqueous form. By entrapping the hydrated hair growth inhibiting agent in or on the sorbent, the antiperspirant composition remains in anhydrous form without negative interaction with the hydrated hair growth inhibiting agent

In accordance with exemplary embodiments, a method for making the antiperspirant composition includes providing silica particles and sorbing a hair growth inhibiting agent with the silica particles. The method further includes mixing the silica particles holding the hair growth reducing agent with antiperspirant ingredients including an active antiperspirant compound to form the antiperspirant composition. Other suitable methods for forming the antiperspirant composition known to those skilled in the art may also be used.

In accordance with another exemplary embodiment, a method for making a skin care composition includes providing sorbent particles and sorbing a hydrated compound with the silica particles. The method further includes mixing the silica particles holding the hydrated compound with an anhydrous base including typical anhydrous skin care components to form the skin care composition. Other suitable methods for forming the skin care composition known to those skilled in the art may also be used.

The following is an example of an antiperspirant composition in accordance with an exemplary embodiment including an invisible solid product. The example is provided for illustration purposes only and is not meant to limit the various embodiments of the antiperspirant product in any way. All materials are set forth in weight percent.

### EXAMPLE 1

### Antiperspirant Product—Invisible Solid Formulation

| Ingredient | Wt. % |
|---|---|
| Cyclopentasiloxane | 32.0 to 46.0 |
| Stearyl alcohol | 15.0 to 21.0 |
| Aluminum Zirconium Pehntachlorohydrex - GLY | 18.0 to 24.0 |
| PPG -14 Butyl Ether | 6.0 to 15.0 |
| Hydrogenated Castor Oil | 2.0 to 5.0 |
| Myristal Myristate | 1.0 to 4.0 |
| Polyethylene | 0.0 to 3.0 |
| Cetyl Alcohol | 0.0 to 3.0 |
| Composition of the Silica Composition Below: | |
| Silica | 0.0 to 6.0 |
| Potassium or Calcium Thioglycolate | 0.0 to 3.0 |
| Aloe Barbadensis | 0.0 to 3.0 |
| Actiphyte of Lavender | 0.0 to 3.0 |
| Total | 100.0 |

It is noted that the antiperspirant composition includes a hydrated compound, i.e., the potassium or calcium thioglycolate, and an anhydrous base formed by the remainder of components. Negative interactions between the hydrated compound and the anhydrous portion of the composition are avoided by sorbing the hydrated compound with the sorbent. Such interactions include undesirable changes to the consistency of the composition, clogging spray or aerosol nozzles, change in texture or feel, formation of crystals, and other known changes.

The hydrated compound may be formed by agents that are not intended or effective for hair growth inhibition. For example, the hydrated compound may be caffeine, vitamin E, jojoba oil, aloe, lavender, extracts, essential oils, minerals, or other hydrated compounds, or combinations thereof. For such embodiments, the hydrated compound is entrapped, encapsulated, or otherwise separated from the anhydrous base by the sorbent. The sorbent may be dissolved, partially dissolved, or otherwise modified to cause desorption or release of the sorbed hydrated compound. Upon desorption or release, the hydrated compound can interact with skin at the application site. Such desorption may occur over time, with the slow release of the sorbed hydrated compound allowing for nearly continuous and delayed interaction of the hydrated compound with the skin.

Accordingly, skin care compositions, such as antiperspirant compositions, which include hydrated compounds in an anhydrous base have been disclosed. Further, antiperspirant compositions that exhibit antiperspirant efficacy, that are effective to reduce or inhibit growth of hair, and that have application efficacy and aesthetics, and methods for making such antiperspirant compositions have been described. Unlike the prior art, the exemplary embodiments taught herein form an antiperspirant composition that comprise a hair growth inhibiting agent that is sorbed by silica particles. During use, the silica particles release the hair growth inhibiting agent over time as they are contacted by moisture generated by the user through perspiration. The antiperspirant composition further includes an active antiperspirant compound effective to inhibit secretion of perspiration and/or accompanying odors.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the processes without departing from the scope defined by the claims, which includes known equivalents and foreseeable equivalents at the time of filing this patent application.

What is claimed is:

1. An antiperspirant composition comprising:
an active antiperspirant compound;
a sorbent dispersed throughout the antiperspirant composition; and
a hair growth inhibiting agent sorbed by the sorbent.

2. The antiperspirant composition of claim 1 wherein the hair growth inhibiting agent is in aqueous form.

3. The antiperspirant composition of claim 1 wherein the hair growth inhibiting agent is in aqueous form and the antiperspirant composition is in non-aqueous form.

4. The antiperspirant composition of claim 1 wherein the composition is in stick, aerosol or roll-on form.

5. The antiperspirant composition of claim 1 wherein the sorbent comprises porous hydrophilic silica particles and the hair growth inhibiting agent is adsorbed onto and absorbed into the porous hydrophilic silica particles.

6. The antiperspirant composition of claim 1 wherein the hair growth inhibiting agent is a hydrated compound and wherein the remainder of the antiperspirant is anhydrous.

7. The antiperspirant composition of claim 1 wherein the sorbent comprises silica particles and the hair growth inhibiting agent is adsorbed onto surfaces of the silica particles, absorbed into pores of the silica particles or a combination thereof.

8. The antiperspirant composition of claim 7 wherein the silica particles are effective to release the hair growth inhibiting agent when contacted with body-generated moisture.

9. The antiperspirant composition of claim 7 wherein the hair growth inhibiting agent is potassium or calcium thioglycolate.

10. The antiperspirant composition of claim 9 wherein the antiperspirant composition comprises: no more than 6.0% by weight of the silica particles and no more than 3.0% by weight of the potassium or calcium thioglycolate.

11. The antiperspirant composition of claim 9 wherein the antiperspirant composition comprises, as percent by weight:

| | |
|---|---|
| Cyclopentasiloxane | 32.0 to 46.0 |
| Stearyl alcohol | 15.0 to 21.0 |
| Aluminum Zirconium Pehntachlorohydrex - GLY | 18.0 to 24.0 |
| PPG - 14 Butyl Ether | 6.0 to 15.0 |
| Hydrogenated Castor Oil | 2.0 to 5.0 |
| Myristal Myristate | 1.0 to 4.0 |
| Polyethylene | 0.0 to 3.0 |
| Cetyl Alcohol | 0.0 to 3.0 |
| Silica | 0.0 to 6.0 |
| Potassium or Calcium Thioglycolate | 0.0 to 3.0 |
| Aloe Barbadensis | 0.0 to 3.0 |
| Actiphyte of Lavender | 0.0 to 3.0. |

12. A skin care composition comprising:
an anhydrous base;
a sorbent dispersed throughout the anhydrous base; and
a hydrated compound sorbed by the sorbent, wherein interaction between the anhydrous base and the hydrated compound is prevented.

13. The skin care composition of claim 12 wherein the hydrated compound comprises a hair growth inhibiting agent.

14. The skin care composition of claim 13 wherein the hair growth inhibiting agent is potassium or calcium thioglycolate.

15. The skin care composition of claim 12 wherein the hydrated compound is selected from caffeine, vitamin E, jojoba oil, aloe, lavender, extracts, essential oils, and minerals.

16. The skin care composition of claim 12 wherein the sorbent comprises silica particles dispersed through the anhydrous base.

17. The skin care composition of claim 12 wherein the sorbent comprises porous hydrophilic silica particles and the hydrated compound is adsorbed onto and absorbed into the porous hydrophilic silica particles.

18. The skin care composition of claim 12 comprising, as percent by weight:

| | |
|---|---|
| Cyclopentasiloxane | 32.0 to 46.0 |
| Stearyl alcohol | 15.0 to 21.0 |
| Aluminum Zirconium Pehntachlorohydrex - GLY | 18.0 to 24.0 |
| PPG -14 Butyl Ether | 6.0 to 15.0 |
| Hydrogenated Castor Oil | 2.0 to 5.0 |
| Myristal Myristate | 1.0 to 4.0 |
| Polyethylene | 0.0 to 3.0 |
| Cetyl Alcohol | 0.0 to 3.0 |
| Silica | 0.0 to 6.0 |
| Potassium or Calcium Thioglycolate | 0.0 to 3.0 |
| Aloe Barbadensis | 0.0 to 3.0 |
| Actiphyte of Lavender | 0.0 to 3.0. |

19. A method for making an antiperspirant composition comprising:
sorbing a hair growth inhibiting agent with a sorbent to form a sorbed hair growth inhibiting agent;
forming an antiperspirant mixture with an active antiperspirant compound; and
dispersing the sorbed hair growth inhibiting agent into the antiperspirant mixture.

20. The method of claim 19 wherein sorbing a hair growth inhibiting agent with a sorbent to form a sorbed hair growth inhibiting agent comprises sorbing potassium or calcium thioglycolate with silica.
